(19) United States
(12) Patent Application Publication
(10) Pub. No.: US 2024/0032570 A1
(43) Pub. Date: Feb. 1, 2024

(54) ORAL FAST-DISINTEGRATING SOLID FOOD AND METHOD FOR MANUFACTURING SAME

(71) Applicants: UHA MIKAKUTO CO., LTD., Yamatokooriyama-shi, Nara (JP); YUMEMIKOUBOU CO., LTD., Ninohe-shi, Iwate (JP)

(72) Inventors: Taiji MATSUKAWA, Yamatokooriyama-shi, Nara (JP); Yasumasa YAMADA, Yamatokooriyama-shi, Nara (JP); Fumito KUBOTA, Ninohe-shi, Iwate (JP)

(73) Assignees: UHA MIKAKUTO CO., LTD., Yamatokooriyama-shi, Nara (JP); YUMEMIKOUBOU CO., LTD., Ninohe-shi, Iwate (JP)

(21) Appl. No.: 18/018,852

(22) PCT Filed: Aug. 17, 2021
(86) PCT No.: PCT/JP2021/030082

§ 371 Date: Jan. 30, 2023

(30) Foreign Application Priority Data
Aug. 18, 2020 (JP) 2020-138257

(51) Int. Cl. A23L 5/00; A23L 33/125; A23G 3/34

(52) U.S. Cl. CPC A23L 5/51; A23L 33/125; A23G 3/0014

(57) ABSTRACT

A solid food including 90 mass % or more of a water-soluble component, wherein the water-soluble component includes 60 mass % or more of carbohydrates when the mass of the solid food is 100 mass %, the solid food has a porosity of 17-35% (inclusive), and the solid food disintegrates in 5-65 seconds (inclusive) according to disintegration testing using an oral fast-disintegration measurement device; and a method for manufacturing a solid food, including steps for obtaining raw material granules using a raw material that includes a carbohydrate and a binder liquid, drying the raw material granules in an environment at a temperature of 10-60° C. (inclusive) to obtain dry granules, compressing the dry granules to obtain a solid compressed article, moistening the solid compressed article to obtain a moistened compressed article, and drying the moistened compressed article in an environment at a temperature of 10-60° C. (inclusive) to obtain a dry compressed article.

### TECHNICAL FIELD

This invention relates to a solid food product that quickly intraorally disintegrates and a method for manufacturing the same.

### BACKGROUND ART

WO2018/229894 discloses confectionery having a grape-like mouthfeel. This confectionery contains a solidified gel composition and a collagen casing coating the solidified gel composition. This confectionery containing the gel composition does not quickly intraorally dissolve.

Since a tablet is not intended to intraorally disintegrate, it does not quickly intraorally dissolve. Thus, the tablet tastes of a flavoring substance applied on its surface.

Patent Document 1: Pamphlet of WO2018/229894

### SUMMARY OF THE INVENTION

### Problems to be Solved by the Invention

An object of this invention is to provide a solid food product that quickly intraorally disintegrates while maintaining sufficient strength during transportation, and a method for manufacturing the same.

### Solutions to the Problems

The above-described problems can be solved by, for example, making a solid food product containing a predetermined amount of a water-soluble sugar have a porosity within a predetermined range.

A first invention of this Description relates to a solid food product containing 90 mass % or more of a water-soluble ingredient.

The solid food product contains 60 mass % or more of a sugar as the water-soluble ingredient when a mass of the solid food product is 100 mass %. The solid food product has a porosity of from 17% to 35%, both inclusive.

In view of this, this solid food product disintegrates in from 5 seconds to 65 seconds, both inclusive, in a disintegration test using an intraoral rapid disintegration measurement device.

An exemplary solid food product is a nutritional supplementary food.

The sugar preferably contains 60 mass % or more of any one or both of saccharides and sugar alcohols with a solubility in 25° C. water of from 20 g/100 g H₂O to 300 g/100 g H₂O, both inclusive, when the mass of the solid food product is 100 mass %. The sugar preferably contains 30 mass % or more of erythritol when the mass of the solid food product is 100 mass %.

The solid food product preferably has a volume of from 0.5 cm³ to 10 cm³ both inclusive, and a thickness of 2 mm or more.

The solid food product preferably has a surface roughness of from 5 μμm to 500 μm, both inclusive.

An invention other than the above herein relates to a method for manufacturing the solid food product. This method includes a granulation step, a first drying step, a compression step, a moistening step, and a second drying step.
The granulation step is a step of obtaining raw material granules using a raw material containing the sugar and a binder liquid. An exemplary ingredient other than the sugar or the binder liquid is a functional ingredient.
The first drying step is a step of drying the raw material granules under an environment at from 10° C. to 60° C., both inclusive, to obtain dried granules.
The compression step is a step of compressing the dried granules to obtain a solid compressed product.
The moistening step is a step of moistening the solid compressed product to obtain a compressed-and-moistened product.
The second drying step is a step of drying the compressed-and-moistened product under an environment at from 10° C. to 60° C., both inclusive, to obtain a compressed-and-dried product.
The solid food product obtained by this method is any one of the solid food products described above.
Specifically, the solid food product is a solid food product that contains 90 mass % or more of a water-soluble ingredient, and the water-soluble ingredient contains 60 mass % or more of a sugar when the mass of the solid food product is 100 mass %.

The binder liquid is preferably a solution with a viscosity of from 10 mPa·s to 1×10³ mPa·s, both inclusive, and a weight ratio obtained by dividing a weight of the sugar in the raw material by a weight of the binder liquid is preferably from 8 to 20, both inclusive.

The raw material granules preferably have a particle diameter (d) represented by a central value of particle diameter distribution of from 0.5 mm to 1.5 mm, both inclusive, a length (t) of from 0.75 mm to 7.5 mm, both inclusive, and an aspect ratio (t/d) of from 1.5 to 5, both inclusive.

The raw material granules preferably have a hardness of from 1 gf to 10 gf, both inclusive.

The compression step is preferably a step of compressing a mixture of the dried granules and a lubricant having a weight of from 0.1% to 5%, both inclusive, of a weight of the dried granules with a compressive force of from 10 kgf/cm² to 300 kgf/cm², both inclusive, to obtain a solid compressed product.

The moistening step is preferably a step of causing the solid compressed product to contain a water content having a weight of from 0.5% to 3%, both inclusive, of a weight of the solid compressed product to obtain a compressed-and-moistened product.

### ADVANTAGEOUS EFFECTS OF THE INVENTION

The invention described herein can provide a solid food product that quickly intraorally disintegrates while maintaining sufficient strength during transportation, and a method for manufacturing the same.

### BRIEF DESCRIPTION OF THE DRAWINGS

FIG. 1 is a flowchart illustrating an exemplary manufacturing process of a solid food product.

FIG. 2 is a photograph substituted for a drawing that illustrates a surface of a solid food product (Example) obtained with a compressive force of 87 kgf/cm².

FIG. 3 is a photograph substituted for a drawing that illustrates a surface of a solid food product (Comparative Example) obtained with a compressive force of 566 kgf/cm².

### DETAILED DESCRIPTION OF THE INVENTION

The following describes embodiments for executing the present invention using the drawings. The present invention is not limited to the embodiments described below, and also encompasses those appropriately modified from the following embodiments within an obvious range by a person skilled in the art.

A first invention herein relates to a solid food product. The solid food product means a food product in a solid form at ordinary temperature. Exemplary solid food products include nutritional supplementary foods, supplements, confectionery, preservative foods, emergency foods, and seasonings. The solid food product is generally preferred to be directly ingested via an oral route. The solid food product may be one that is dissolved in a solvent (for example, water) and ingested. The solid food product is preferred to contain a functional ingredient. The solid food product may be a medicine or a drug, or may be a quasi-drug.

The solid food product contains 90 mass % or more of a water-soluble ingredient when the mass of the solid food product is 100 mass %. Containing 90 mass % or more of the water-soluble ingredient ensures reducing a grainy texture when the solid food product is put in an oral cavity. The water-soluble ingredient means an ingredient that dissolves in water at an ordinary temperature (25° C.). Examples of the water-soluble ingredient include water-soluble sugars. Examples of the sugar include saccharides, oligosaccharides, polysaccharides, and sugar alcohols. The saccharides include monosaccharides and disaccharides. The solid food product contains 60 mass % or more of a sugar as the water-soluble ingredient when the mass of the solid food product is 100 mass %. The water-soluble ingredient may also include those in an emulsified state and those in an insoluble microparticle state with a largest diameter of 10 microns or less, besides those that dissolve in water. The water-soluble ingredient may include an ingredient that does not provide a grainy texture in a mouth.

The water-soluble sugar preferably has a solubility in 25° C. water of from 20 g/100 g H₂O to 300 g/100 g H₂O, both inclusive, and may have a solubility in 25° C. water of from 20 g/100 g H₂O to 250 g/100 g H₂O, both inclusive, or from 50 g/100 g H₂O to 200 g/100 g H₂O, both inclusive. Examples of such a sugar include one type or a mixed sugar of two or more types of glucose, galactose, mannose, fructose, lactose, sucrose, maltose, erythritol, palatinose, xylitol, sorbitol, and reduced palatinose. As shown in Example 3, the solid food product with lactose as a main sugar may remain grainy in a mouth, and therefore, it is preferred that the solid food product contains a sugar other than lactose as a main ingredient or that the solid food product does not contain lactose. Sorbitol has high solubility and is not easily molded. Therefore, it is preferred that the solid food product contains a sugar other than sorbitol as a main ingredient or that the solid food product does not contain sorbitol.

The solid food product is preferred to contain 60 mass % or more (or 70 mass % or more, 80 mass % or more, 90 mass % or more) of any one or both of saccharides and sugar alcohols as the above-described water-soluble sugar when the mass of the solid food product is 100 mass %. The solid food product is preferred to contain 30 mass % or more (or 40 mass % or more, 50 mass % or more, 60 mass % or more, 70 mass % or more, 80 mass% or more, 90 mass% or more) of erythritol when the mass of the solid food product is 100 mass%. As proven by the Examples, those containing erythritol as a main sugar are excellent in moldability and solubility.

The solid food product is preferred to contain a functional ingredient. The functional ingredient is preferred to be 0.1 mass % or more and 35 mass % or less, and may be 0.1 mass % or more and 10 mass % or less, 0.1 mass % or more and 1 mass % or less, or 1 mass % or more and 5 mass % or less when the mass of the solid food product is 100 mass %. Examples of the functional ingredient include vitamins, minerals, lactic acid bacteria, amino acids, peptides, proteins, lipids, nucleic acids, antioxidant substances, flavoring agents, and various kinds of active ingredients. Since the solid food product containing the functional ingredient quickly intraorally disintegrates, the concentration of the functional ingredient can be intraorally increased to increase an intraoral absorption rate of the functional ingredient, and thus, an instant result is expected. Such examples include vitamins. Those containing the functional ingredient are particularly effective as a functional food for intraoral dryness, such as xerostomia and Sjögren syndrome.

The solid food product has a porosity of 17% or more and 35% or less. In view of this, this solid food product disintegrates in 5 seconds or more and 65 seconds or less in a disintegration test using an intraoral rapid disintegration measurement device. This solid food product is expected to quickly intraorally disintegrates basically without disintegrating in a transportation phase. In view of this, the solid food product has a porosity in an appropriate range while containing much water-soluble sugar as described above. The porosity only needs to be appropriately adjusted taking the type and the proportion of the sugar contained in the solid food product into account. The porosity may be 17.5% or more and 34.5% or less, 17.5% or more and 26% or less, 25% or more and 27% or less, 24.5% or more and 26.5% or less, or 30% or more and 34.5% or less.

The solid food product is in any shape. Examples of the shape of the solid food product include a columnar shape (tablet shape) without corners, a quadrangular-prismatic shape without corners, a spherical shape, an elliptical spherical shape, and a polygonal-prismatic shape without corners. The solid food product preferably has a volume of 0.5 cm³ or more and 10 cm³ or less, and a thickness of 2 mm or more. The volume of the solid food product may be 1 cm³ or more and 5 cm³ or less, 1 cm³ or more and 40 cm³ or less, 0.5 cm³ or more and 4 cm³ or less, 3 cm³ or more and 10 cm³ or less, 4 cm³ or more and 10 cm³ or less, or 5 cm³ or more and 10 cm³ or less. The thickness only needs to be a height when the solid food product is laid most stably when it is laid on a floor. The thickness of the solid food product only needs to be an appropriate thickness taking the above-described volumes into account. An exemplary thickness of the solid food product is 2 mm or more, and may be 3 mm or more, 5 mm or more, or 1 cm or more. The thickness may be 3 mm or less, 5 mm or less, 1 cm or less, or 2 cm or less.

A weight per solid food product only needs to be appropriately adjusted. An exemplary weight per solid food product is 0.1 g or more and 10 g or less, and may be 0.5 g or more and 5 g or less, or 1 g or more and 5 g or less.

The solid food product preferably has a surface roughness of 5 μm or more and 500 μm or less, and may have a surface roughness of 20 μm or more and 400 μm or less, 50 μm or more and 450 μm or less, 100 μm or more and 3500 μm or less, or 150 μm or more and 300 μm or less. The surface roughness only needs to be obtained based on the surface roughness specified in, for example, ISO 25178. When the surface roughness is small, the surface area is small, and thus, the solid food product is less likely to disintegrate. Meanwhile, when the surface roughness is large, the moldability is poor, and thus, the solid food product is likely to disintegrate.

The solid food product of the present invention disintegrates in 5 seconds or more and 65 seconds or less in the disintegration test using the intraoral rapid disintegration measurement device. This is a test imitating an inside of an oral cavity and means that the solid food product disintegrates in 5 seconds or more and 65 seconds or less in an oral cavity of a normal healthy subject. Disintegration means that the solid food product becomes lumps of a predetermined size or less. A preferable example of the disintegrating period is 7 seconds or more and 65 seconds or less, and the disintegrating period is preferably 10 seconds or more and 65 seconds or less, is preferably 60 seconds or less, is preferably 55 seconds or less, is preferably 40 seconds or less, is preferably 35 seconds or less, and is preferably 30 seconds or less. Adjusting the amount of sugar, the porosity, the amount of functional ingredient, and the molding method ensures adjusting the disintegrating period. A tablet that contains an ordinary disintegrant dissolves via a lot of water content. In view of this, a large amount of water content is necessary for intraorally dissolving the tablet, and thus, thirst is caused. Meanwhile, the solid food product of the present invention quickly disintegrates with a small amount of water content (saliva), and thus, thirst is not caused. When the solid food product contains a flavoring agent, the flavoring agent quickly intraorally dissolves out, and thus, the aroma quickly spreads.

An invention other than the above herein relates to a method for manufacturing the solid food product.

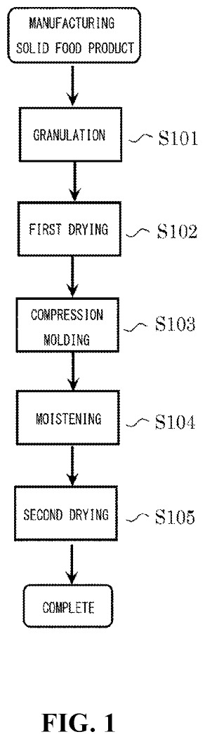

FIG. 1 is a flowchart illustrating an exemplary manufacturing process of the solid food product.

As illustrated in FIG. 1, this method includes a granulation step (S101), a first drying step (S102), a compression step (S103), a moistening step (S104), and a second drying step (S105). The solid food product obtained in this method is any one of the solid food products described above.

Specifically, the solid food product is a solid food product that contains 90 mass % or more of a water-soluble ingredient, and the water-soluble ingredient contains 60 mass % or more of a sugar when the mass of the solid food product is 100 mass %.

The granulation step (S101) is a step for obtaining raw material granules using a raw material containing the sugar and a binder liquid. An exemplary ingredient other than the sugar or the binder liquid is a functional ingredient.

An exemplary binder liquid is a solution with a viscosity of 10 mPa·s or more and 1 ×10³ mPa·s or less, and the viscosity may be 10 mPa·s or more and 5×10² mPa·s or less, 10 mPa·s or more and 1×10² mPa·s or less, 1×10² mPa·s or more and 5×10² mPa·s or less, or 1×10² mPa·s or more and 1×10³ mPa·s or less. Examples of a binder (for example, in a powder form) that constitutes the binder liquid include any one or a mixture of two or more types of ethyl cellulose, hypromellose, hydroxypropyl methylcellulose, and hydroxypropyl cellulose (HPC). Among them, the hydroxypropyl cellulose (HPC) is preferred. A weight ratio obtained by dividing the weight of the sugar in the raw material by the weight of the binder liquid is preferred to be 8 or more and 20 or less, and may be 10 or more and 20 or less, 15 or more and 20 or less, 8 or more and 15 or less, or 10 or more and 15 or less.

The raw material granules preferably have a particle diameter (d) represented by a central value of the particle diameter distribution of 0.5 mm or more and 1.5 mm or less, a length (t) of 0.75 mm or more and 7.5 mm or less, and an aspect ratio (t/d) of 1.5 or more and 5 or less. The raw material granules are preferred to have a hardness of 1 gf or more and 10 gf or less, and may have a hardness of 3 gf or more and 10 gf or less, 3 gf or more and 6 gf or less, or 5 gf or more and 10 gf or less.

The granulation step (S101) is preferably a method that makes the shapes of the raw material granules fall within a certain range. An example of a specific method is extrusion granulation. Uniforming the shapes of the raw material granules into, for example, a columnar shape ensures obtaining a solid compressed product with a low compressive force, thereby ensuring dispersed voids. While small voids are dispersed when ordinary granulated products are compression molded, uniforming the granules into a certain large size allows molding with a low compressive force, which not only increases the porosity in the molded product but also allows making large lumps of voids instead of the dispersed voids. Moreover, increasing the strength of the granules ensures preventing the voids from being filled as the granules do not break before they are put into a molding machine and maintain the shapes, and furthermore, ensures preventing the voids from being reduced caused by the granules broken at an early stage in the compression process. For giving the hardness to the molded product, as described later, since a large part of the raw material is the water-soluble ingredient, a part of the surface of the molded product is dissolved by being moistened and dried, and thus, practical strength can be secured.

In the case of a tablet press generally used in a compression molding machine, the weight of the molded product depends on the volume of powder or granules filled in a die. In the present invention, it is preferred to secure the porosity by uniforming the shapes of the granules, and since the optimum range of the porosity is small, it is preferred to reduce a variation in the filling weight of the granules. In order to reduce the variation in the filling weight of the granules, it is effective to uniform the sizes of the granules and make the sizes of the granules smaller than the size of the die. Accordingly, it is preferred that the ratio of the length of an opening (a surface that intakes the granules) of the die to the particle diameter of the granules does not exceed 3:1. For example, the lengths of the granules filled in a circular die with a diameter of 15 mm are preferred to be 5 mm or less. When the lengths of the granules are 1 mm, an exemplary aspect ratio, the ratio of the particle diameter to the length of the granules, is 5. The length of the granules is preferably 3 mm or less, and then, the aspect ratio is 3. When the weight variation is reduced, it is preferred to approximate the length of the granules to the particle diameter; however, it is difficult to secure the voids in the molded product when the size of the granules itself is too small, and therefore, at least an aspect ratio of 1 or more where the major axis and the minor axis of the granule are the same is preferred. The granule shape and size when the voids in the molded product are stably secured have a major axis of ⅓ or less of the diameter of the opening of a die hole, and an aspect ratio of the granules of 1 to 5, preferably 1.5 to 5.

It is preferred that the granules obtained by granulation maintain the shapes until they are put into the molding machine. Furthermore, the voids cannot be secured in case the granules are broken at an early stage of the compression process by the molding machine; however, the granules need to be broken at the end of the compression process, otherwise the molded product itself does not become hard. Accordingly, the granules preferably have optimum hardness, and to give the hardness, the viscosity of the binder liquid of a water-soluble polymer added during the granulation is important. The hardness of the granules is preferred to be 1 to 10 gf, and the viscosity of the binder liquid added in order to obtain this hardness is 10 to 1000 mPa·s.

The first drying step (S102) is a step of drying the raw material granules under an environment at 10° C. or more and 60° C. or less to obtain dried granules. The temperature and the drying period only need to be adjusted as necessary. Since the raw material granules are thus dried at a low temperature, the dried granules can be obtained without losing functions of the functional ingredient. A preferred exemplary drying temperature is 20° C. or more and 60° C. or less, and may be 25° C. or more and 60° C. or less, 30° C. or more and 55° C. or less, or 25° C. or more and 50° C. or less. An exemplary drying period is 10 minutes or more and 2 days or less, and may be 1 hour or more and 1 day or less, 2 hours or more and 15 hours or less, 3 hours or more and 12 hours or less, or 4 hours or more and 8 hours or less.

The compression step (S103) is a step of compressing the dried granules to obtain a solid compressed product. The compression step is preferred to be a step of compressing a mixture of the dried granules and a lubricant having a weight of 0.1% or more and 5% or less of the weight of the dried granules with a compressive force of 10 kgf/cm² or more and 300 kgf/cm² or less to obtain the solid compressed product.

The moistening step (S104) is a step of moistening the solid compressed product to obtain a compressed-and-moistened product. The moistening step is preferred to be a step of causing the solid compressed product to contain a water content having a weight of 0.5% or more and 3% or less of the weight of the solid compressed product to obtain the compressed-and-moistened product. The more the moisture ratio is, the more the strength of the molded product is, but the disintegrating period in a mouth also lengthens, and therefore, the moisture ratio is preferably adjusted. The moisture ratio for making an intraoral quick dissolving period equal to or less than 60 seconds is 3% or less, and the moisture ratio for making the intraoral quick dissolving period equal to or less than 30 seconds is 2% or less. Since a practical hardness of 2 kgf or more can be obtained with a moisture ratio of 0.5% or more, the moisture ratio (weight) is 0.5 to 3%, is preferably 0.5 to 2%, and is further preferably 0.5 to 1%. Since the molding takes place in such a condition with a little water content, the case where the functions of the functional ingredient are lost can be effectively prevented. The moistening step is preferably performed in an environment with high temperature and moisture. An exemplary temperature in the moistening step is from 40° C. to 100° C., both inclusive, and may be from 50° C. to 90° C., both inclusive, from 60° C. to 90° C., both inclusive, from 40° C. to 80° C., both inclusive, from 50° C. to 75° C., both inclusive. An exemplary moisture in the moistening step is from 40% RH to 100% RH, both inclusive, and may be from 50% RH to 90% RH, both inclusive, from 60% RH to 90% RH, both inclusive, from 40% RH to 80% RH, both inclusive, from 50% RH to 75% RH, both inclusive. The moistening period only needs to be adjusted as necessary depending on the humidity and the like. An exemplary moistening period is from 1 second to 1 hour, both inclusive, and may be from 2 seconds to 10 minutes, both inclusive, from 3 seconds to 5 minutes, both inclusive, from 5 seconds to 3 minutes, both inclusive, and from 5 seconds to 1 minute, both inclusive.

The second drying step (S105) is a step of drying the compressed-and-moistened product under an environment at 10° C. or more and 60° C. or less to obtain a compressed-and-dried product. The temperature and the drying period only need to be adjusted as necessary. An exemplary preferable drying temperature is from 20° C. to 60° C., both inclusive, and may be from 25° C. to 60° C., both inclusive, from 30° C. to 55° C., both inclusive, from 25° C. to 50° C., both inclusive. An exemplary drying period is from 10 minutes to 2 day, both inclusive, and may be from 1 hour to 1 day, both inclusive, from 2 hours to 15 hours, both inclusive, from 3 hours to 12 hours, both inclusive, or from 4 hours to 8 hours, both inclusive.

The obtained compressed-and-dried product may directly be the solid food product. The obtained compressed-and-dried product is sterilized by heat and packed so as to be the solid food product.

### EXAMPLES

### Test Example 1

Strength Test of Granules (n=5)

Complying with JIS Z 8841 and using a particle hardness tester, NEW GRANO, manufactured by OKADA SEIKO CO., LTD., a breaking terminal was pressed against the granules at a speed of 5 μm/s to measure a load when it broke.

### Test Example 2

Measurement of Aspect Ratio (Length/Particle Diameter) of Granules (n=10)

Using a microscope VHX-970F (50 times) manufactured by KEYENCE CORPORATION, a length (minor axis) and a major axis were measured to calculate an aspect ratio.

### Test Example 3

Measurement of Porosity of Molded Product (n=3)

A porosity of the molded product was calculated from the following formula.

Porosity of molded product (%)=((volume of molded product−volume of powder)/volume of molded product)×100

The volume of the molded product was calculated from a measurement value of the dimension. The volume of the powder was calculated from measurement values of a particle density and a weight by a densimeter, AccuPyc 1330, manufactured by Micromeritics Instrument Corporation.

### Test Example 4

Strength Test of Molded Product (n=5)

Complying with JIS Z 8841 and using a load cell type tablet hardness tester PC-30 manufactured by OKADA SEIKO CO., LTD., a breaking terminal was pressed against the molded product at a speed of 0.5 mm/s to measure a load when it broke.

### Test Example 5

Disintegration Test of Molded Product (n=3)

The molded product was put between two metal plates having a mesh 2.1 mm square, artificial saliva at 37° C. was dropped onto the molded product from a position at a height of 80 mm at a constant speed of 6 mL/min, and a period it took until the molded product broke to leave no residue was measured using a measuring device for oral disintegration, Tricorptester, manufactured by OKADA SEIKO CO., LTD. The detection of the disintegration period is a mechanism that senses the contact of the upper and lower metal plates with a sensor after the molded product breaks to leave no residue on the metal plates with the meshes. The artificial saliva was obtained by adding 1.47 g of potassium chloride, 1.44 g of sodium chloride, and 3 g of polysorbate 80 to 1 L of distilled water and dissolving them.

### Test Example 6

Shape Evaluation of Molded Product Surface (n=3)

Using a one-shot 3D shape measurement system VR-5000 manufactured by KEYENCE CORPORATION, a depth of a trough from a baseline was measured by profile measurement of a surface of the molded product, and the maximum value was presented complying with ISO 25178.

### Example 1

### Manufacturing Process and Characteristic Values (Manufacturing Process, Compression Molding Process, and Moistening and Drying Process of Granules)

After mixing 700 g of erythritol and 300 g of L-ascorbic acid with an all-around mixing stirrer 5DM manufactured by DALTON Corporation for 3 minutes, 46 g of a 7.5%HPC-L solution (viscosity 220 mPa·s) was added and mixed for 3 minutes. After extrusion granulating the mixed powder with a cylindrical granulator HG-300V (hole diameter 0.8 mm) manufactured by HATA TEKKOSHO CO., LTD., it was dried for one night at 50° C. with a forced-air box dryer CDP-12-9.6WDS manufactured by OKADA SEIKO CO., LTD.

After passing the granules obtained by drying through a 10 mesh screen, calcium stearate whose weight corresponds to 1% of the weight of the granules was added, and they were mixed. The mixture was molded with a compressive force of 171 kgf/cm² with a target weight of the molded product of 1 g using a circular punch with a diameter of 15 mm by a single punch tablet press N-30E manufactured by OKADA SEIKO CO., LTD. After moistening (increase weight 1%) the obtained compression molded product by leaving it in a thermo-hygrostat at 70° C. and 80%RH for 10 seconds, it was dried for one night at 40° C. in a forced-air dryer to obtain the molded product.

The granules had a diameter of 0.8 mm, an average length of 2.5 mm, an aspect of 3.1, and a granule hardness of 4 gf. The molded product had a diameter of 15 mm, a thickness of 5.02 mm, a volume of 0.86 cm³, a hardness of 4.2 kgf, a porosity of 23.2%, and a disintegrating period of 17 seconds.

### Example 2

Relation between Compressive Force and Porosity, and Disintegrating Period

Using the single punch tablet press, the granules to which the calcium stearate had been added and which had been mixed already, obtained in Embodiment 1, were manufactured by varying a compressive force. The obtained molded products were moistened and dried similarly to Example 1. The measurement results of porosities and disintegrating periods of the obtained molded products are shown in the table.

According to the results, the porosity was reduced as the compressive force increased and the disintegrating period was lengthened, and the disintegrating period was 60 seconds or less with 17.9% or more porosity.

TABLE 1

| | Examples | | | Comparative examples | |
|---|---|---|---|---|---|
| Compressive force | 41 | 171 | 253 | 318 | 472 |
| (kgf/cm²) | | | | | |
| Porosity (%) | 25.7 | 19.8 | 17.9 | 16.5 | 14.9 |
| Disintegrating period (s) | 18 | 25 | 55 | 83 | 125 |

### Example 3

### Comparison of Saccharides in VC10% Additive System (Adjusting Compressive Force and Porosity)

In the granulation step, 900 g of sugar alcohols/saccharides, and 100 g of L-ascorbic acid were taken, and 70 g of a 5%HPC-L solution (viscosity 80 mPa.$) was added as a binder liquid. In the compression molding process, the compressive force was adjusted so as to have approximately the same porosities. The table shows the evaluation results of the characteristic values of the molded products (already undergone a moistening and drying process) obtained with other conditions according to Example 1.

According to the results, the disintegrating periods of the tested sugar alcohols/saccharides were 60 seconds or less. In the case of the use of lactose, the grainy texture remains in a mouth, and therefore, the use of lactose is not preferred from the aspect of the mouthfeel.

TABLE 2

| | | | | | Unpreferable |
|---|---|---|---|---|---|
| Sugar alcohols/ | Examples | | | | example |
| saccharides | Erythritol | Xylitol | Sucrose | Glucose | Lactose |
| Compressive | 115 | 146 | 118 | 119 | 133 |
| force | | | | | |
| (kgf/cm²) | | | | | |
| Porosity (%) | 25.1 | 25.5 | 26.5 | 26.3 | 23.7 |
| Hardness (kgf) | 3.2 | 3.3 | 3.1 | 3.2 | 3.1 |
| Disintegrating | 24 | 27 | 34 | 36 | 32 |
| period (s) | | | | | |

Grainy texture in mouth

### Example 4

### Viscosity (HPC-L, M) of Binder Liquid and Granule Hardness, and Properties of Molded Product

In the granulation step, 900 g of erythritol and 100 g of L-ascorbic acid were taken, and granulated by varying concentrations and kinds of a water-soluble polymer (HPC) in the binder liquid. In the compression molding process, the compressive force was adjusted so as to have approximately the same porosities. The table shows the evaluation results of the characteristic values of the molded products (already undergone a moistening and drying process) obtained with other conditions according to Example 1.

According to the results, as the viscosity of the binder liquid increased, the obtained granule hardness increased, and therefore, the hardness of the molded product after the compression also increased. It was possible to use a low-viscosity type (HPC-L) and a medium-viscosity type (HPC-M) of HPC in combination for the viscosity adjustment, and approximately the same granules and molded product were obtained. In preparing a high-viscosity binder liquid, the usage of the HPC-M can be increased.

Also in a blending composition with a highly water-soluble ingredient, the granulation with water alone made fragile granules, these fragile granules failed to be solidified even though they were compression molded, and therefore, the molded product was not obtained. In view of this, the binder (of a water solution with the binder liquid having a viscosity of 10 mPa·s or more) of a water-soluble polymer is preferred in the granulation.

TABLE 3

| | | L. Low- | | | | M. Medium- | |
|---|---|---|---|---|---|---|---|
| Kinds of HPC | Water | viscosity type | | | | viscosity type | |
| Concentration of HPC solution (%) | 0 | 2.5 | 5 | 7.5 | 10 | 1.0 | 2.0 |
| Viscosity of binder liquid (mPa · s) | 1 | 10 | 80 | 220 | 720 | 20 | 200 |
| Saccharides binder liquid (-) | 12.9 | 12.9 | 12.9 | 13.8 | 13.8 | 12.9 | 13.8 |
| Granule hardness (gf) | Unmeasurable | 1.7 | 1.7 | 3.3 | 8.0 | 1.8 | 3.1 |
| | due to | | | | | | |
| | fragile granules | | | | | | |
| Compressive force (kgfm²) | 100 to 300 | 121 | 115 | 120 | 114 | 115 | 117 |
| Porosity (%) | Molded product | 25.2 | 25.1 | 25.1 | 25.8 | 25.3 | 25.4 |
| | unobtainable | | | | | | |
| | due to | | | | | | |
| | fragility | | | | | | |
| Hardness of molded product | — | 0.8 | 0.9 | 1.7 | 2.2 | 0.8 | 1.8 |
| after compression (kgf) | | | | | | | |
| Hardness of molded product | — | 3.0 | 3.2 | 3.3 | 4.0 | 3.3 | 3.5 |
| after hardening (kgf) | | | | | | | |
| Disintegrating period of molded | — | 9 | 16 | 20 | 25 | 17 | 21 |
| product after hardening (s) | | | | | | | |

### Example 5

### Degree of Moisture vs Strength, Disintegrating Period

The manufacturing method followed that of Example 1. In the granulation step, 900 g of erythritol and 100 g of L-ascorbic acid were taken, 70 g of a 5%HPC-L solution (saccharides/binder liquid ratio: 12.9) was added as a binder liquid, granulation was performed, and thereafter, the granules were dried. In the compression molding process, the conditions were uniformed so as to have approximately the same compressive force (115 kgf/cm²) and porosity (25%). The obtained compression molded product was put and moistened in a thermo-hygrostat at 70° C. and 80%RH, taken out at regular intervals, and thereafter, was dried for one night at 40° C. in a forced-air dryer, and thus, the molded product was manufactured. The weights before and after the moistening of the molded product were measured, and the increased weight ratios were used as moisture ratios.

According to the results, as the moisture ratio (moistening period) increased, the hardness and the disintegrating period of the molded product also increased. The disintegrating periods were 60 seconds or less when the moisture ratios were 3% or less. The practical hardness of the molded product is 3 to 6 kgf, and excessive moisture delays the disintegrating period.

TABLE 4

| Moistening period (s) | 10 | 20 | 30 | 40 | 60 | 80 |
|---|---|---|---|---|---|---|
| Moisture ratio (%) | 0.7 | 1.3 | 1.8 | 2.3 | 3.0 | 4.0 |
| Hardness (kgf) | 3.1 | 4.5 | 5.5 | 6.1 | 6.9 | 7.6 |
| Disintegrating period (s) | 21 | 26 | 30 | 38 | 52 | 65 |

### Example 6

### Exemplary Improvement in Taste by Using Saccharides in Combination (Usage of Erythritol 30%)

In the granulation step, 350 g of erythritol, 450 g of glucose, and 200 g of L-ascorbic acid were taken, 70 g of a 5%HPC-L solution (saccharides/binder liquid ratio: 11.4) was added as a binder liquid, granulation was performed, and thereafter, the the granules were dried. Calcium stearate whose weight corresponded to 1% of the weight of the obtained granules was added, and compression molding took place with a compressive force of 120 kgf/cm². The obtained molded product provided satisfactory characteristic values with a porosity of 25%, a hardness of 3.5 kgf, and a disintegrating period of 19 seconds.

Using the glucose in combination increased sweetness compared with the case where the saccharide was the erythritol alone, and thus, the acidity of the L-ascorbic acid could be reduced. The functional ingredients with strong tastes, such as the L-ascorbic acid, can have the tastes adjusted (masked) by combining the saccharides.

### Example 7

### Validation 1 in Actual Production Scale

Manufacturing in a 20 kg scale was performed with four kinds of compounding rates shown in the table. The raw materials excluding HPC-L, fine silicon dioxide, a flavoring agent, and calcium stearate were mixed with a new speed kneader NSK-650 S model manufactured by OKADA SEIKO CO., LTD. for 3 minutes, thereafter, an HPC-L solution was added, and they were mixed for 3 minutes. The mixed powder was extruded and granulated with the cylindrical granulator HG-300V (hole diameter 0.8 mm) manufactured by HATA TEKKOSHO CO., LTD., and thereafter, was dried for one night at 50° C. with the forced-air box dryer CDP-12-9.6WDS manufactured by OKADA SEIKO CO., LTD.

The dried and obtained granules were passed through a 10 mesh screen, thereafter, a flavoring agent, fine silicon dioxide, and calcium stearate were added, and they were mixed. The mixture was continuously tableted (compression molded) for 10 minutes with a target weight of the molded product of 1 g using a circular (chamfered portion square edges) punch with a diameter of 15 mm by a rotary tablet press LIBRA2 (20 rpm) manufactured by KIKUSUI SEISAKUSHO LTD. The obtained compression molded product was moistened by being left in a thermo-hygrostat at 70° C. and 80%RH for 10 seconds, and thereafter, was dried for one night at 40° C. in a forced-air dryer to obtain the molded product.

In the manufacturing method of the present invention, no tableting failure was recognized in any tested blending compositions, no external appearance failure, such as a crack and a chip, was present, and the manufacturing could be continuously performed.

TABLE 5
Vitamin C

| | |
|---|---|
| Erythritol | 64.874% |
| Vitamin C | 31.834% |
| Vitamin B2 | 0.066% |
| HPC-L | 0.500% |
| Stevia | 0.701% |
| Fine silicon dioxide | 0.425% |
| Flavoring agent | 0.600% |
| Calcium stearate | 1.000% |
| Total water-soluble ingredient | 97.975% |

Zinc/maca

| | |
|---|---|
| Erythritol | 88.47% |
| Citric acid | 3.00% |
| Zinc gluconate | 4.10% |
| Black maca extract | 1.53% |
| Fine silicon dioxide | 0.30% |
| HPC-L | 0.50% |
| Flavoring agent | 0.90% |
| Calcium stearate | 1.20% |
| Total water-soluble ingredient | 96.07% |

TABLE 6
Iron/folic acid

| | |
|---|---|
| Erythritol | 92.206% |
| Citric acid | 3.000% |
| Ferric pyrophosphate | 2.100% |
| Stevia | 0.080% |
| Folic acid | 0.014% |
| HPC-L | 0.700% |
| Fine silicon dioxide | 0.100% |
| Flavoring agent | 0.800% |
| Calcium stearate | 1.000% |
| Total water-soluble ingredient | 96.000% |

Lactic acid bacteria

| | |
|---|---|
| Erythritol | 93.49% |
| Citric acid | 3.00% |
| Lactic acid bacteria | 1.00% |
| (heated fungus body) | |
| HPC-L | 0.70% |
| Flavoring agent | 0.81% |
| Calcium stearate | 1.00% |
| Total water-soluble ingredient | 97.19% |

### Example 8

### Validation 2 in Actual Production Scale

The manufacturing conditions and the evaluation results of the characteristic values in Example 7 are as shown in the table.

TABLE 7

| | | | | Iron/folic | Lactic acid |
|---|---|---|---|---|---|
| Sample name | | Vitamin C | Zinc/maca | acid | bacteria |
| Compounding | Erythritol (%) | 64.9 | 88.5 | 92.2 | 93.5 |
| properties | Total water-soluble ingredient (%) | 98.0 | 96.1 | 96.0 | 97.2 |
| | Viscosity of binder liquid (mPa · s) | 530 | 200 | 720 | 720 |
| | Saccharides/binder liquid ratio (—) | 11.8 | 12.6 | 13.2 | 13.4 |
| Manufacturing | Compressive force (kgf/cm²) | 113 | 68 | 85 | 87 |
| conditions | Moisture ratio (%) | 1.0 | 0.8 | 1.1 | 0.9 |
| Property | Aspect ratio (—) | 2.3 | 3.0 | 2.6 | 2.6 |
| evaluations | Granule hardness (gf) | 2.0 | 3.1 | 1.8 | 6.7 |
| of granules | | | | | |
| Property | Weight (g) | 0.997 | 1.000 | 1.000 | 0.985 |
| evaluations of | Thickness (mm) | 5.02 | 5.85 | 5.80 | 5.99 |
| molded product | Volume (cm³) | 0.86 | 1.00 | 0.99 | 1.03 |
| | Particle density (g/cm³) | 1.5162 | 1.4543 | 1.4686 | 1.4515 |
| | Porosity (%) | 23.2 | 31.5 | 31.5 | 34.0 |
| | Molded product hardness (kgf) | 4.2 | 6.5 | 3.8 | 4.4 |
| | Disintegrating period (s) | 17 | 39 | 32 | 16 |

### Example 9

### Surface Shape of Molded Product

Depths of pores on the molded product surfaces of the “lactic acid bacteria” and the “iron/folic acid” obtained by the blending composition and the manufacturing method described in Example 7 were measured by the method in Test Example 6. In addition, the molded product obtained by increasing the compressive force was also measured as a comparison example. The maximum values of the compressive forces, the molded product porosities, the disintegrating periods, and the depths of pores on the surfaces are put together in the table.

TABLE 8

| | | Example | | Comparative example | |
|---|---|---|---|---|---|
| | | Lactic acid | Iron/folic | Lactic acid | Iron/folic |
| Sample name | | bacteria | acid | bacteria | acid |
| Manufacturing | Compressive force | 87 | 85 | 566 | 566 |
| conditions | (kgf/cm³) | | | | |
| | Moisture ratio (%) | 0.9 | 1.1 | 0.9 | 1.1 |
| Physical property | Porosity (%) | 33.9 | 32.2 | 14.6 | 13.8 |
| evaluations | Disintegrating period (s) | 18 | 10 | 180 or more | 180 or more |
| | Pore depth (μm) | 263 | 190 | 10 | 6 |

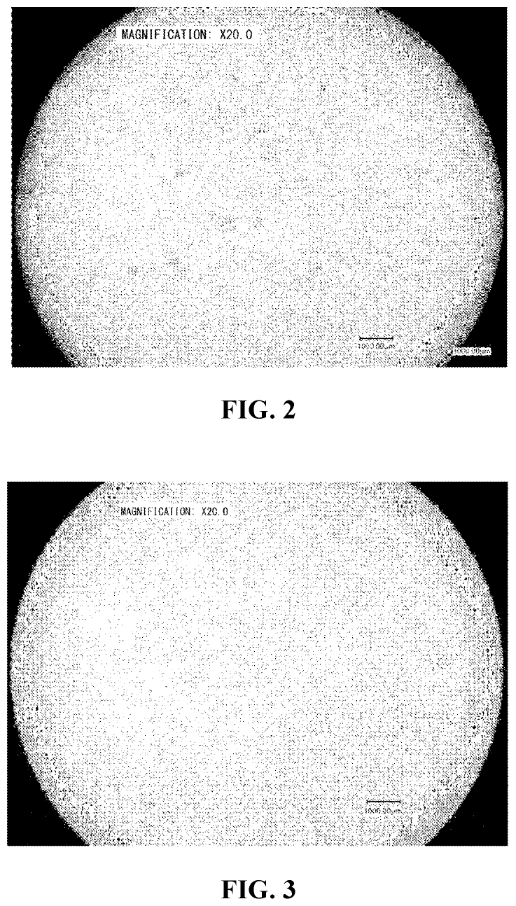

As a representative example, the surface photograph (20 times) of the “lactic acid bacteria” molded product is exemplarily illustrated. FIG. 2 is a photograph substituted for a drawing illustrating the surface of a solid food product (Example) obtained with a compressive force of 87 kgf/cm². FIG. 3 is a photograph substituted for a drawing illustrating the surface of a solid food product (Comparative Example) obtained with a compressive force of 566 kgf/cm². From FIG. 2 and FIG. 3, differences in conditions of the unevenness of the surfaces and voids caused by the compressive force are seen, and it is seen that the low compressive force maintains granule shapes. Since the granules quickly disintegrate after the molded product disintegrates with saliva, the one made with the small compressive force has higher disintegrativity.

### INDUSTRIAL APPLICABILITY

This invention may be used in, for example, a food product industry.

1.-6. (canceled)

7. A method for manufacturing a solid food product containing 90 mass % or more of a water-soluble ingredient, the water-soluble ingredient containing 60 mass % or more of a sugar when a mass of the solid food product is 100 mass %, the method comprising:
a granulation step of obtaining raw material granules using a raw material containing the sugar and a binder liquid;
a first drying step of drying the raw material granules to obtain dried granules;
a compression step of compressing the dried granules to obtain a solid compressed product;
a moistening step of moistening the solid compressed product to obtain a compressed-and-moistened product; and
a second drying step of drying the compressed-and-moistened product to obtain a compressed-and-dried product, wherein the raw material granules have a particle diameter (d) represented by a central value of particle diameter distribution of from 0.5 mm to 1.5 mm, both inclusive, a length (t) of from 0.75 mm to 7.5 mm, both inclusive, an aspect ratio (t/d) of from 1.5 to 5, both inclusive, and a hardness of from 1 gf to 10 gf, both inclusive.

8. The method for manufacturing the solid food product according to claim 7, wherein the binder liquid is a solution with a viscosity of from 10 mPa·s to 1×10³ mPa·s, both inclusive, and a weight ratio obtained by dividing a weight of the sugar in the raw material by a weight of the binder liquid is from 8 to 20, both inclusive.

9. (canceled)

10. (canceled)

11. The method for manufacturing the solid food product according to claim 7, wherein the compression step is a step of compressing a mixture of the dried granules and a lubricant having a weight of from 0.1% to 5%, both inclusive, of a weight of the dried granules with a compressive force of from 10 kgf/cm² to 300 kgf/cm², both inclusive, to obtain a solid compressed product.

12. The method for manufacturing the solid food product according to claim 7, wherein the moistening step is a step of causing the solid compressed product to contain a water content having a weight of from 0.5% to 3%, both inclusive, of a weight of the solid compressed product to obtain a compressed-and-moistened product.

13. The method for manufacturing the solid food product according to claim 7, wherein the solid food product is a nutritional supplementary food having a surface roughness of from 20 μm to 400 μm, both inclusive.

14.-18. (canceled)

19. The method for manufacturing the solid food product according to claim 12, wherein the moistening step is a step performed at from 60° C. to 90° C., both inclusive, in from 5 seconds to 20 seconds, both inclusive.

20. The method for manufacturing the solid food product according to claim 12, wherein the moistening step is a step performed at from 60° C. to 90° C., both inclusive, in from 5 seconds to 10 seconds, both inclusive.

21. The method for manufacturing the solid food product according to claim 7, wherein the second drying step is a step performed in from 4 hours to 8 hours, both inclusive.

22. The method for manufacturing the solid food product according to claim 13, wherein the second drying step is a step performed in from 4 hours to 8 hours, both inclusive.

23. The method for manufacturing the solid food product according to claim 7, wherein the binder liquid is a solution with a viscosity of from 10² mPa·s to 1×10³ mPa·s, both inclusive.

24. The method for manufacturing the solid food product according to claim 7, wherein the binder liquid is a solution with a viscosity of from 80 mPa·s to 1×10³ mPa·s, both inclusive. (Original) The method for manufacturing the solid food product according to claim 7, wherein the binder liquid is a solution with a viscosity of from 10² mPa·s to 5×10² mPa·s, both inclusive.

26. The solid food product produced by the method for manufacturing the solid food product according to claim 7.

27. The solid food product according to claim 26, wherein the solid food product is a nutritional supplementary food, a supplement, or confectionery.

28. The solid food product produced by the method for manufacturing the solid food product according to claim 23.

29. The solid food product according to claim 28, wherein the solid food product is a nutritional supplementary food, a supplement, or confectionery.

30. The solid food product produced by the method for manufacturing the solid food product according to claim 24.

31. The solid food product according to claim 30, wherein the solid food product is a nutritional supplementary food, a supplement, or confectionery.

32. The solid food product produced by the method for manufacturing the solid food product according to claim 25.

33. The solid food product according to claim 32, wherein the solid food product is a nutritional supplementary food, a supplement, or confectionery.
